(19) United States
(12) Patent Application Publication
(10) Pub. No.: US 2005/0177083 A1
(43) Pub. Date: Aug. 11, 2005

(54) Foot eversion inhibitor

(76) Inventor: Arlan Dean Heil, Houston, TX (US)

Correspondence Address: Guy McClung, No. 347, 16690 Champion Forest Drive, Spring, TX, 77379-7023, US

(21) Appl. No.: 10/774,901

(22) Filed: Feb. 9, 2004

(51) Int. Cl. A61F 5/00

(52) U.S. Cl. 602/27

(57) ABSTRACT

Apparatuses and methods for inhibiting foot eversion, the apparatus, in at least certain aspects, including a cuff positionable around a leg, a strap with a first end and a second end, the first end connected to the cuff and the second end connectible to footwear at a location on an outer side of the footwear.

### BACKGROUND OF THE INVENTION

1. Field of the Invention

This invention is directed to apparatus for inhibiting the eversion of a foot during standing, walking, or running and to methods for using such an apparatus.

2. Description of Related Art

The prior art discloses a wide variety of ankle braces, orthopedic shoes, drop foot supports, and foot inversion and eversion inhibitors. Typically these devices attempt to address problems associated with drop foot, unwanted foot inversion or introversion (movement of the foot—or inward falling of ankle)—inwardly, or unwanted foot eversion (movement of the foot—or ankle)—outwardly. Many of the prior art devices are complex, cumbersome, non-adjustable, difficult to make and install, and uncomfortable to the wearer.

There has long been a need for apparatus that effectively inhibits foot eversion. There has long been a need for such a apparatus that is easily made and easily installed. There has long been a need for such apparatus which is not unduly uncomfortable for the wearer. There has long been a need for such apparatus that can flex and have a range of motion to accommodate movement of the wearer's leg, ankle, and foot.

### SUMMARY OF THE PRESENT INVENTION

In one aspect, the present invention discloses an apparatus for inhibiting foot eversion, the apparatus including a cuff positionable around a leg above an ankle joint, a strap with a first end and a second end, the first end connected to the cuff and the second end connectible to footwear at a location on a portion of the footwear at an outer side thereof. The present invention, in one embodiment, discloses apparatus for inhibiting foot eversion. In one aspect, the apparatus includes footwear, e.g. a shoe or boot. In one aspect, the apparatus includes a cuff that releasably wraps around a person's leg over, near or above the ankle and has a strap extending down from the cuff to an outer part of footwear, e.g. a shoe, at which point the strap is permanently or releasably connected to the shoe, either interiorly or exteriorly of the shoe. Optionally, a front member is permanently or releasably connected to the cuff at one end and to a front part of the shoe (e.g. but not limited to a tongue of the shoe) at the other end. Suitable fasteners and/or VELCRO™ material or other releasably-cooperating hook-loop fastener material (“RC Material”) may be used for connection of the strap and front member to the cuff and to the shoe. In one particular aspect, the cuff is positioned immediately above outwardly protruding parts of an ankle, with a lower part of the cuff on said parts, so that these ankle parts prevent the cuff from moving lower down on the leg of which the ankle is a part and also so that this positioning allows the ankle, via the cuff, to provide support to the outer side of the foot to inhibit eversion or a tendency or condition of the foot to flop, fall or move outwardly.

In one aspect a shoe is used which is originally manufactured with cloth on its tongue and/or on its interior that releasably cooperates with RC material on the strap and/or on the front member. Many commercially available shoes are made with such material. The cuff can be made of any suitable flexible soft material and, in one aspect, includes cushion material for comfort. The strap may have RC material on both sides (as may the front member) and the cuff may have corresponding material on its surfaces.

The present invention, in certain embodiments, discloses a foot eversion inhibitor according to the present invention that is combined with aspects of a support system [in certain aspects, similar to that disclosed in U.S. Pat. No. 6,007,506 and in U.S. application Ser. No. 09/746,262 filed 14/22/2000, now abandoned, both co-owned with the present invention and fully incorporated herein for all purposes] that has a belt, cuff or strip that is releasably secured around a person's lower leg and one or more support posts that are releasably connected to and/or held by the belt, etc. The support post(s) extends from the belt strip down to a boot, sandal, or shoe worn on the person's foot. A lower portion of the support post is releasably or permanently connected to part of the shoe. In another aspect two such support posts are used, one on each side of the foot near the heel.

In certain embodiments, the present invention discloses: a system for inhibiting foot eversion, the foot joined to the leg by an ankle joint, the leg having a lower leg portion above the ankle joint, the ankle joint having bony portions that project from either side of the ankle, the system having a strap that extends down from a cuff positioned around the leg above the ankle and extending down to a part of the shoe an outer (e.g. left part of the left shoe) and in front of the ankle bones.

As discussed and shown in detail below, any of the straps, front members, and/or support posts or members described herein may be either permanently or releasably attached or connected to the inside or outside of footwear and/or cuff. Straps, support posts, and/or front members may be attached or connected to footwear and/or to a cuff with any known fastener material, fastener, sewing and/or adhesive (e.g. glue).

It is, therefore, an object of at least certain preferred embodiments of the present invention to provide:
New, useful, unique, efficient, nonobvious apparatuses for inhibiting foot eversion, and, in certain aspects, footwear with such apparatus;
Such apparatus which is flexible and is releasably connected to a person's leg and footwear so that it accommodates movement of the wearer while still providing needed eversion inhibition;
Such apparatus which is not unduly uncomfortable to the wearer;
Such apparatus which is easily applied and easily removed;
Such apparatus which is useful with certain commercially available footwear without changing the footwear or adding any additional structure or device thereto.

Certain embodiments of this invention are not limited to any particular individual feature disclosed here, but include combinations of them distinguished from the prior art in their structures and functions. Features of the invention have been broadly described so that the detailed descriptions that follow may be better understood, and in order that the contributions of this invention to the arts may be better appreciated. There are, of course, additional aspects of the invention described below and which may be included in the subject matter of the claims to this invention. Those skilled in the art who have the benefit of this invention, its teachings, and suggestions will appreciate that the conceptions of this disclosure may be used as a creative basis for designing other structures, methods and systems for carrying out and practicing the present invention. The claims of this invention are to be read to include any legally equivalent devices or methods which do not depart from the spirit and scope of the present invention.

The present invention recognizes and addresses the previously-mentioned problems and long-felt needs and provides a solution to those problems and a satisfactory meeting of those needs in its various possible embodiments and equivalents thereof. To one of skill in this art who has the benefits of this invention's realizations, teachings, disclosures, and suggestions, other purposes and advantages will be appreciated from the following description of preferred embodiments, given for the purpose of disclosure, when taken in conjunction with the accompanying drawings. The detail in these descriptions is not intended to thwart this patent's object to claim this invention no matter how others may later disguise it by variations in form or additions of further improvements.

### DESCRIPTION OF THE DRAWINGS

A more particular description of embodiments of the invention briefly summarized above may be had by references to the embodiments which are shown in the drawings which form a part of this specification. These drawings illustrate certain preferred embodiments and are not to be used to improperly limit the scope of the invention which may have other equally effective or legally equivalent embodiments.

FIG. 1A is a side view of an apparatus according to the present invention. FIG. 1B is an exploded view of the apparatus of FIG. 1A. FIGS. 1C, 1D, 1E and 1F are side views of parts of the apparatus of FIG. 1A.

FIG. 2 is a perspective view of a system according to the present invention.

FIG. 3 is a perspective view of a system according to the present invention.

### DESCRIPTION OF EMBODIMENTS PREFERRED AT THE TIME OF FILING FOR THIS PATENT

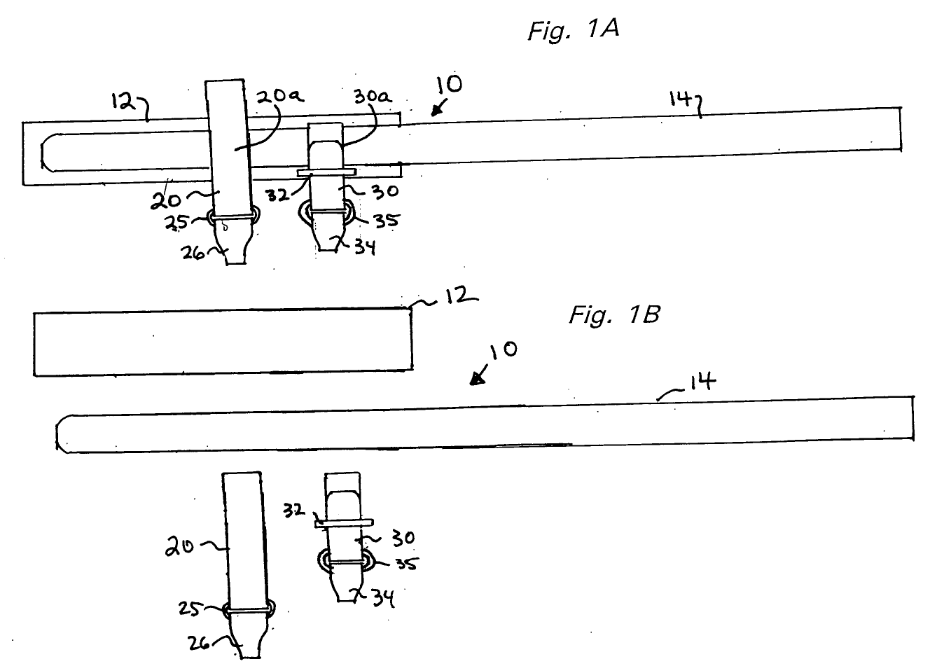

FIGS. 1A-1F show an apparatus 10 according to the present invention or parts thereof.

The apparatus 10 has a cuff 12 to which is connected a wrap member 14. The wrap member 14 and the cuff 12 have or are made of RC material so that the wrap member 14 is releasably connected to the cuff 12; but it is within the scope of this invention (as is the case for the strap and front member described below) for the wrap member to be sewn, stapled, glued, and/or riveted to the cuff 12.

The wrap member 14 passes through an eyelet 21 formed by part of a strap 20 and through an opening 31 formed between portions of an optional front member 30.

The apparatus 10 as shown in FIG. 1A is not yet installed on a leg. The wrap member 14 may be sufficiently long, as shown in FIG. 1A to wrap around a leg and over portions 20a of the strap 20 and portion 30a of the front member 30 providing further securement of the strap 20 and the front member 30 and protecting them from accidental contact or release.

Both the strap 20 and the front member 30 may be a fixed, nonadjustable length; e.g. when an apparatus 10 is custom made for a single individual. As shown, however, in FIG. 1A the position of the strap 20 and the front member 30 are adjustable along the length of the wrap member 14 and may be positioned at any desired location along this length and they are both also adjustable in length (vertical dimension as shown in FIG. 1A) to facilitate installation and to render them most effective—the strap 20 connected to the cuff 12 inhibiting foot eversion and the optional front member 30 in reducing the effects of drop foot.

By using flexible, yet sufficiently strong, material for the strap 20 and the front member 30, a foot within footwear having an apparatus 10 is permitted a sufficient amount of freedom of movement so that normal activities—e.g. walking and running—are not impeded, yet inhibition of foot eversion and of drop foot are accomplished.

In one particular aspect the strap 20 is located so that it does not ride on a projecting portion of an ankle joint.

A shown in FIGS. 1C and 1D, the strap 20 has a portion 20b that wraps around the wrap member 14 and part of which abuts the cuff 12. Optionally an opening 23 is formed using, e.g., a rivet 24 and/or RC material to provide a convenient holding area to hold the strap 20 and to pull on it to adjust the length of the strap 20, e.g. to insure that there is sufficient tightness of the strap 20 when it is in use to inhibit foot eversion. A lower portion 24 of the strap 20 passes through a metal loop 25 and a lowermost part 26 of the strap 20 also has a portion that passes through the metal loop 25. This lowermost portion 26 is connected, releasably or permanently, interiorly or exteriorly, to an outer part of footwear used with the apparatus 10. In one aspect the lowermost portion 26 is riveted on the outside of footwear. In another aspect, the lowermost portion 26 is RC material which releasably mates with corresponding RC material on either the inside or on the outside of footwear, in one aspect with the metal loop 25. In one particular aspect the lowermost portion 26 is permanently attached to footwear and the strap 20 is permanently attached to the cuff 12 so that the apparatus 10 remains with that particular footwear.

In one aspect when the cuff 12, wrap member 14 and strap 20 all are made of or incorporate appropriately located RC material, the length of the strap 20 is adjustable by adjusting the portion 20b with respect to the wrap member 14. As shown in FIG. 1D, length adjustment can be effected by releasing a portion 20d from a portion 20e and pulling upon the part with the opening 23 or, alternatively, by pulling the strap 20 down with respect to the portion 20e. Thus, in one aspect, a foot may be inserted into footwear with an apparatus 10, the cuff 12 is installed around a leg with the strap 20 relatively loose; then, by pulling up using the opening 23, the strap 20 is tightened as desired; and then, if desired, the wrap member 14 may be wrapped around the cuff 12 covering a portion of the strap 20. Similarly, when a front member 30 is present, its length can be adjusted after the cuff 12 is positioned around a leg.

As shown in FIGS. 1E and 1F a front member 30 includes a selectively releasable clasp 32 (e.g. like any such well-known openable/closable clasp—used, e.g., in suspenders) which is openable to adjust the length of the front member 30 and then closable (shown closed in FIG. 1F) to hold the front member 30 securely so that the desired length is maintained. Alternatively any releasable buckle apparatus or slide-bar buckle apparatus may be used, e.g. with buckle parts connected to the footwear (and may be used with the front member also). Such a clasp or buckle provides strap or front member length adjustability. With the clasp opened, pulling on a top end 33 tightens the front member 30 which is connected to an outer front part of the footwear [used with the apparatus 10] forward of a vertical line from the ankle joint to the footwear's bottom. A lower part 34 of the front member 30 is connected to a front part of the footwear and a metal loop 35 between the main portion of the front member 30 and the lower part 34 permits the length adjustment described above. A clasp member also may be used with a strap or front member made of RC material.

Optionally, the strap 20 and/or the front member 30 are single pieces of RC material which are releasably connected to corresponding RC material on the cuff 12 and/or on the wrap member 14 and, optionally, the other ends of the strap 20 and/or front member 30 are similarly releasably connected to either corresponding cloth of the footwear itself or to RC material connected to the footwear. A sufficiently long strap 20 and front member 30 provide length adjustment simply by position selection and final positioning of the strap 20 and the front member 30.

The strap 20's lower end is connected to footwear at a location adjacent to or forward of an ankle joint on an outer side of footwear (or, alternatively, at an outer side of the footwear but within the footwear to an inside part thereof) to inhibit foot eversion. In certain aspects this lower connection point is on the footwear's outer side (i.e., a left side of a left foot or a right side of a right foot) adjacent a foot's arch, adjacent the ball of a foot, or adjacent a little toe. The location of this connection with respect to the height of the footwear is, in certain aspects: at or near the sole of the footwear; at or near a lace eyelet of the footwear; or at a point between the lace eyelets and the sole. One, two, or more straps may be used with lower ends connected at different points on the footwear.

In certain aspects the cuff 12 is installed above an ankle joint around a leg. In other aspects the cuff 12 is installed around the ankle joint, with or without an opening through which projecting parts of the ankle joint can protrude. An apparatus 10 as shown in FIG. 1A is usable with either left footwear or right footwear. The strap 20 may employ the clasp 32 shown used with the front member 30 and the clasp 32 may be deleted from the front member 30 and it may be structured like the strap 20.

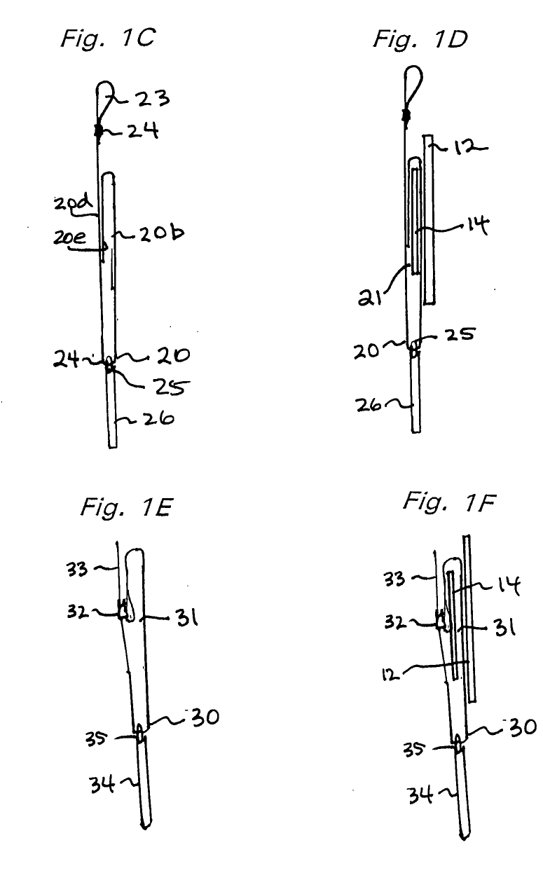

FIG. 2 illustrates a shoe 38 according to the present invention with an apparatus 40 according to the present invention which has a cuff 42 which may be either wrapable around a leg L or which may be closed but sufficiently expandable to insert the foot F through it. An optional front member 43 functions as does the front members previously described and as shown is made of RC material that has a top end connected to the cuff 42 and a lower end that releasably mates with corresponding RC material 39 on top of the shoe 38. Alternatively, the front member 43 may be connected in the interior of the shoe 38. Optionally the cuff 42 includes a layer of cushion material 42a.

A strap 44 has a top end connected to the cuff 42 and a lower end connected with a rivet 45a to an outer portion 46a of the shoe 38. Optionally, an additional strap 47 has a top end connected to the cuff 42 and a lower end connected to an outer portion 46b of the shoe 38 with a rivet 45b. Both straps 44 and 47 may be made of flexible material, including, but not limited to, RC material and/or VELCRO™ material. It is within the scope of this invention for any two items of any embodiment herein (e.g. strap to cuff; front member to shoe; strap to footwear) to be connected using selectively releasable snap apparatus and/or rivets.

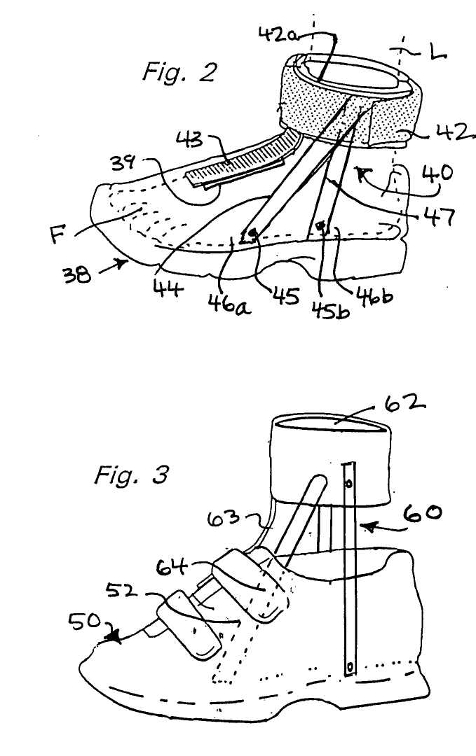

FIG. 3 shows a shoe 50 according to the present invention with an apparatus 60 according to the present invention which has an optional front member 63 with a lower end releasably connected to a front part of the shoe 50 and an upper end connected to a cuff 62 which is configured for encircling a leg. A strap 64 has a top end connected to the cuff 62 and a lower portion extending inside the shoe 50 and connected to an interior part 52 of the shoe 50. In one aspect the strap 64 is made of RC material and the interior part 52 is such that the RC material of the strap 64 releasably mates with it or the part 52 is itself RC material that corresponds to and releasably mates with the RC material of the strap 64.

The apparatus 60 has two optional relatively rigid side struts 65 which each has a top end connected to the cuff 62 and a bottom end connected to the shoe 50. These side struts provide rigidity and foot support and may be connected closer to the rear of the shoe 50 than as shown in FIG. 3 as desired. Optionally either strut may be deleted. These struts may be made of any suitable sufficiently rigid and strong material, e.g., but not limited to, aluminum or plastic; and, in certain aspects, these struts are as disclosed in U.S. Pat. No. 6,007,506.

The present invention, therefore, provides in some, but not in necessarily all, embodiments an apparatus for inhibiting foot eversion, the apparatus including a cuff positionable around a leg above an ankle joint, a strap with a first end and a second end, the first end connected to the cuff and the second end connectible to footwear at a location on a portion of the footwear at an outer side thereof. Such an apparatus may have one or some, in any possible combination, of the following: a front member having a top end connectible to the cuff and a bottom end connectible to a front part of the footwear; wherein the second end of the strap is connectible to an outside part of the footwear; wherein the second end of the strap is releasably connectible to the footwear; at least one side post connectible between the cuff and the footwear on a side of the footwear at a rear portion of the footwear; the at least one side post comprising two side posts, one on each side of a rear part of the footwear adjacent a heel thereof; a strap for wrapping around the cuff to secure the cuff around a wearer's leg; an amount of releasably cooperating fastener material on a surface of the cuff, and a corresponding first amount of releasably cooperating fastener material on a surface of the strap for facilitating securement of the strap to the cuff; a second amount of releasably cooperating fastener material on a surface of the strap for releasable securement thereto of at least part of the first amount of releasably cooperating fastener material; wherein the cuff is positionable above protruding parts of the ankle thereby preventing the cuff from moving down on the leg below the ankle; wherein the cuff is at least partially supported by the protruding parts of the ankle to facilitate inhibition of eversion of the foot; adjustability means for adjusting length of the strap between the cuff and the footwear; wherein the adjustability means is from the group consisting of openable-closable clasp and buckle apparatus; and/or wherein the cuff includes cushion material for the comfort of a user of the apparatus.

The present invention, therefore, provides in at least some embodiments, an apparatus for inhibiting foot eversion, the apparatus including a cuff positionable around a leg above an ankle joint, two straps each with a first end and a second end, the first end connected to the cuff and the second end connectible to footwear at a location on a portion of the footwear at an outer side thereof.

The present invention, therefore, provides in at least some embodiments, an apparatus for inhibiting foot eversion, the apparatus including a cuff positionable around a leg above an ankle joint, at least one strap with a first end and a second end, the first end releasably connected to the cuff and the second end releasably connectible to footwear at a location on a portion of the footwear at an outer side thereof, the second end of the strap releasably connectible to an outside part of the footwear, a strap for wrapping around the cuff to secure the cuff around a wearer's leg, an amount of releasably cooperating fastener material on a surface of the cuff, and a corresponding first amount of releasably cooperating fastener material on a surface of the strap for facilitating securement of the strap to the cuff, wherein the cuff is positionable above protruding parts of the ankle thereby preventing the cuff from moving down on the leg below the ankle, wherein the cuff is at least partially supported by the protruding parts of the ankle to facilitate inhibition of eversion of the foot, and adjustability apparatus for adjusting length of the strap between the cuff and the footwear.

The present invention, therefore, provides methods for inhibiting foot eversion, the methods including emplacing the foot in footwear, the foot wear having an apparatus for inhibiting foot eversion as any described herein according to the present invention, positioning the cuff of such apparatus around the leg above an ankle joint thereof, and connecting an end of a strap thereof to an outer side of the footwear. In such a method the apparatus may include a front member having a top end connectible to the cuff and a bottom end connectible to a front part of the footwear, the method further including connecting the top end of the front member to the cuff and the bottom end of the front member to a front part of the footwear; and any such method may include positioning a cuff of such apparatus above protruding parts of the ankle thereby preventing the cuff from moving down on the leg below the ankle, and at least partially supporting the cuff with the protruding parts of the ankle to facilitate inhibition of eversion of the foot. In any such method, the apparatus may include adjustability apparatus for adjusting length of a strap between the cuff and the footwear, the method further including adjusting the length of the strap with the adjustability apparatus.

In conclusion, therefore, it is seen that the present invention and the embodiments disclosed herein and those covered by the appended claims are well adapted to carry out the objectives and obtain the ends set forth. Certain changes can be made in the subject matter without departing from the spirit and the scope of this invention. It is realized that changes are possible within the scope of this invention and it is further intended that each element or step recited in any of the following claims is to be understood as referring to all equivalent elements or steps. The following claims are intended to cover the invention as broadly as legally possible in whatever form it may be utilized. The invention claimed herein is new and novel in accordance with 35 U.S.C. § 102 and satisfies the conditions for patentability in § 102. The invention claimed herein is not obvious in accordance with 35 U.S.C. § 103 and satisfies the conditions for patentability in § 103. This specification and the claims that follow are in accordance with all of the requirements of 35 U.S.C. § 112.

1. An apparatus for inhibiting foot eversion, the apparatus comprising
a cuff positionable around a leg above an ankle joint,
a strap with a first end and a second end, the first end connected to the cuff and the second end connectible to footwear at a location on a portion of the footwear at an outer side thereof.

2. The apparatus of claim 1 further comprising
a front member having a top end connectible to the cuff and a bottom end connectible to a front part of the footwear.

3. The apparatus of claim 1 wherein the second end of the strap is connectible to an outside part of the footwear.

4. The apparatus of claim 1 wherein the second end of the strap is releasably connectible to the footwear.

5. The apparatus of claim 1 further comprising
at least one side post connectible between the cuff and the footwear on a side of the footwear at a rear portion of the footwear.

6. The apparatus of claim 5 further comprising
the at least one side post comprising two side posts, one on each side of a rear part of the footwear adjacent a heel thereof.

7. The apparatus of claim 1 further comprising
a strap for wrapping around the cuff to secure the cuff around a wearer's leg.

8. The apparatus of claim 1 further comprising
an amount of releasably cooperating fastener material on a surface of the cuff, and
a corresponding first amount of releasably cooperating fastener material on a surface of the strap for facilitating securement of the strap to the cuff.

9. The apparatus of claim 8 further comprising
an second amount of releasably cooperating fastener material on a surface of the strap for releasable securement thereto of at least part of the first amount of releasably cooperating fastener material.

10. The apparatus of claim 1 wherein the cuff is positionable above protruding parts of the ankle thereby preventing the cuff from moving down on the leg below the ankle.

11. The apparatus of claim 10 wherein the cuff is at least partially supported by the protruding parts of the ankle to facilitate inhibition of eversion of the foot.

12. The apparatus of claim 10 further comprising
adjustability means for adjusting length of the strap between the cuff and the footwear.

13. The apparatus of claim 12 wherein the adjustability means is from the group consisting of openable-closable clasp and buckle apparatus.

14. The apparatus of claim 12 wherein the cuff includes cushion material for the comfort of a user of the apparatus.

15. An apparatus for inhibiting foot eversion, the apparatus comprising
a cuff positionable around a leg above an ankle joint,
two straps each with a first end and a second end, the first end connected to the cuff and the second end connectible to footwear at a location on a portion of the footwear at an outer side thereof.

16. An apparatus for inhibiting foot eversion, the apparatus comprising
a cuff positionable around a leg above an ankle joint,
at least one strap with a first end and a second end, the first end releasably connected to the cuff and the second end releasably connectible to footwear at a location on a portion of the footwear at an outer side thereof, the second end of the strap releasably connectible to an outside part of the footwear,
a strap for wrapping around the cuff to secure the cuff around a wearer's leg,
an amount of releasably cooperating fastener material on a surface of the cuff, and
a corresponding first amount of releasably cooperating fastener material on a surface of the strap for facilitating securement of the strap to the cuff,
wherein the cuff is positionable above protruding parts of the ankle thereby preventing the cuff from moving down on the leg below the ankle,
wherein the cuff is at least partially supported by the protruding parts of the ankle to facilitate inhibition of eversion of the foot, and
adjustability means for adjusting length of the strap between the cuff and the footwear.

17. A method for inhibiting foot eversion, the method comprising
emplacing the foot in footwear, the footwear having an apparatus for inhibiting foot eversion, the apparatus comprising a cuff positionable around a leg above an ankle joint, a strap with a first end and a second end, the first end connected to the cuff and the second end connectible to footwear at a location on a portion of the footwear at an outer side thereof, and
positioning the cuff around the leg above an ankle joint thereof, and
connecting the second end of the strap to an outer side of the footwear.

18. The method of claim 17 wherein the apparatus further comprises a front member having a top end connectible to the cuff and a bottom end connectible to a front part of the footwear, the method further comprising
connecting the top end of the front member to the cuff and the bottom end of the front member to a front part of the footwear.

19. The method of claim 17 further comprising
positioning the cuff above protruding parts of the ankle thereby preventing the cuff from moving down on the leg below the ankle, and
at least partially supporting the cuff with the protruding parts of the ankle to facilitate inhibition of eversion of the foot.

20. The method of claim 17 wherein the apparatus further comprises adjustability means for adjusting length of the strap between the cuff and the footwear, the method further comprising
adjusting the length of the strap with the adjustability means.
